(19) United States
(12) Patent Application Publication
(10) Pub. No.: US 2022/0062213 A1
(43) Pub. Date: Mar. 3, 2022

(54) METHODS AND COMPOSITIONS OF SOLABEGRON FOR REDUCING BODY FAT

(71) Applicant: Jubilant Pharma Holdings Inc., Yardley, PA (US)

(72) Inventor: Indranil Nandi, Yardley, NJ (US)

(73) Assignee: Jubilant Pharma Holdings Inc., Yardley, PA (US)

(21) Appl. No.: 17/463,731

(22) Filed: Sep. 1, 2021

Related U.S. Application Data
(60) Provisional application No. 63/073,733, filed on Sep. 2, 2020.

(51) Int. Cl. A61K 31/196; A61K 9/08; A61K 9/00; A61K 47/02; A61K 47/26; A61K 47/18; A61K 47/10; A61K 47/12

(52) U.S. Cl. CPC A61K 31/196; A61K 9/08; A61K 9/0019; A61K 47/12; A61K 47/26; A61K 47/183; A61K 47/10; A61K 47/02

(57) ABSTRACT

Provided herein are compositions and methods useful in the non-surgical removal of fat deposits in patients in need thereof using β3-adrenoreceptor agonists. The invention further provides a method of treating double chin disorder, benign symmetric lipomatosis, adiposis dolorosa, lipedema, and familial partial lipodystrophy in patients in need thereof by administration of solabegron or its pharmaceutically acceptable salts, esters, solvates, polymorphs, enantiomers, or mixtures thereof. The treatment method as per the present invention comprises treatment with solabegron or its pharmaceutically acceptable salts, esters, solvates, polymorphs, enantiomers, or mixtures thereof suitable for injection administration. The compositions and treatment method as per the present invention have advantages of simple preparation, simple and convenient application, easy absorption, and better effect of treating without requiring any surgical procedure.

### FIELD OF THE INVENTION

The present invention relates to methods and compositions of β3-adrenoreceptor agonists for reducing body fat. In particular, but without restriction to the particular embodiments hereinafter described in accordance with the best mode of practice, the present invention provides injection dosage forms of β3-adrenoreceptor agonists such as solabegron and processes for preparing these compositions. The present invention provides application of injection pharmaceutical compositions of solabegron or its pharmaceutically acceptable salts, esters, solvates, polymorphs, enantiomers, or mixtures thereof for non-surgical removal of fat deposits in patients in need thereof.

### BACKGROUND OF THE INVENTION

Increased body weight, obesity, and excess body fat have become a common problem in the modern life. For many people excess body fat is also a source of social distress and reduced self-esteem. Moreover, this can be a cause of other diseases and cosmetic disturbance and may further lead to increased risk of morbidity and mortality due to its associated chronic diseases such as metabolic diseases, hypertension, diabetes, cardiovascular diseases, hyperglycemia, gallbladder diseases, and even some types of cancer.

Fat accumulation on a particular portion of the body is another phenomenon which can occur in individuals who may or may not be overweight. Fat accumulation can involve undesired fat deposits on body portions such as the chest, breast, waist, thighs, buttocks, abdomen, arms, and/or chin. Submental fullness (double chin disorder) is one of such kind of problem wherein fat is accumulated in an individual under the chin. Such localized deposits of fat in an individual may result due to various reasons such as hormonal imbalance, side-effects of medications, or the like.

As a treatment option for fat removal or reduction and in order to improve physical appearance, both surgical and non-surgical methods are available. Surgical procedures include liposuction or lipoplasty in which fat is removed through a cannula inserted into skin connected to a suction source. However, such surgical procedures are costly and can involve a variety of complications and risks, during surgery as well as post-surgery, like skin damage, infection, fluid imbalance, and even death in some cases.

Under non-surgical methods, the first line of treatment is to offer diet and lifestyle advice to patients such as reducing the fat content of their diet and increasing their physical activity. However, many patients find this difficult and need additional help from drug therapy to maintain results from these efforts. Thus, there is a long felt need for other efficient alternative approaches to overcome fat accumulation. Therefore, there is a need for a new approach in the field of pharmaceutical formulations and methods for administration for reducing body fat.

Solabegron is one of the β3-adrenergic receptor agonists, which is under clinical trials for the treatment of overactive bladder and irritable bowel syndrome. Solabegron is chemically known as 3′-[(2-{[(2R)-2-(3-chlorophenyl)-2-hydroxyethyl]amino}ethyl)amino]-[1,1′-biphenyl]-3-carboxylic acid. It is represented with the following chemical structure:

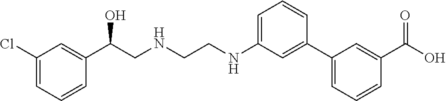 [SMILES: O=C(O)c1cccc(-c2cccc(NCCNC[C@H](O)c3cccc(Cl)c3)c2)c1]

U.S. Pat. No. 6,251,925 assigned to Glaxo Wellcome discloses solabegron as a compound. This patent discloses various salts of solabegron and also discloses that beta-adrenoceptor agonists have been found to be particularly useful as anti-obesity agents and as anti-diabetic agents. The patent publications provide a listing of numerous routes of administration, dose, dosage forms, and excipients that may be included in the dosage form along with the numerous compounds disclosed. The patent makes a series of unsupported claims about the use of beta-adrenoceptor agonists. For example, the patent proposed that the compounds can be used to treat a wide variety of conditions including “hyperglycaemia, obesity, hyperlipemia, irritable bowel syndrome and its associated pain, motility dysfunction, excessive gastrointestinal secretion, non-specific diarrhea, neurogenic inflammation, regulation of intraocular pressure, triglyceridemia, diabetes, e.g. non-insulin-dependent diabetes mellitus (NIDDM or Type 2), such as obese NIDDM and non-obese NIDDM, diabetic complications such as retinopathy, nephropathy, neuropathy, cataracts, coronary heart diseases and arteriosclerosis, osteoporosis; and gastrointestinal disorders, particularly inflammatory gastrointestinal disorders.” The patent also proposed that beta-adrenoceptor agonists may be of use in increasing high-density-lipoprotein (HDL) cholesterol concentration and decreasing triglyceride concentration in blood serum, especially human blood serum, and are therefore for potential use in the treatment and/or prophylaxis of atherosclerosis. The patent also proposes that the compounds may be useful for the treatment of hyperinsulinaemia, depression. muscle wasting, urinary incontinence and wound-healing. However, the patent does not teach application of beta-adrenoceptors agonists for removing or reducing fat.

U.S. Pat. No. 9,907,767 assigned to Velicept Therapeutics discloses the use of solabegron for treating overactive bladder and the symptoms associated such as urinary urgency, frequency of micturitions, nocturia, and urgency urinary incontinence.

Use of β3-adrenergic receptor agonists in the treatment of obesity and metabolic disease is extensively studied in the literature. However, till date no β3-adrenergic receptor agonist is approved in the USA market for the treatment of localized fat reduction and conditions such as double chin disorder, benign symmetric lipomatosis, adiposis dolorosa, lipedema, familial partial lipodystrophy. Moreover, the determination of critical parameters associated with drug development needs to be successfully optimized such as selection of suitable dose, suitable dosage form, suitable route of administration, suitable dosing regimen, and effect in animal and humans. Thus, there is a need to provide an effective drug delivery system of a β3-adrenergic receptor agonist, particularly solabegron to reduce body fat in a patient in need thereof.

Oral dosage forms always remain a first choice of treatment, however, use of an oral dosage form in the treatment of reduction of localized fat and conditions such as double chin disorder, benign symmetric lipomatosis, adiposis dolorosa, lipedema, familial partial lipodystrophy may not be a suitable approach due to a number of factors such as frequent dosage administration, long treatment period, patient compliance and low bioavailability. Injectable formulations may provide a higher level of bioavailability after intramuscular or subcutaneous administration compared to the oral route. The increase in bioavailability of an injectable formulation can provide therapeutic plasma concentration levels with a dose that can be administered intramuscularly or subcutaneously. The total injected dose can be considerably lower than the oral daily dose required over the same time period, thus reducing toxicity and improving patient compliance.

The inventor of the present invention proposes injection dosage forms of solabegron for the treatment of reduction of localized fat and/or obesity-related rare disorders such as double chin disorder, benign symmetric lipomatosis, adiposis dolorosa, lipedema, familial partial lipodystrophy.

### SUMMARY OF THE INVENTION

The present invention relates to β3-adrenergic receptor agonists such as solabegron or its pharmaceutically acceptable salts, esters, solvates, polymorphs, enantiomers, or mixtures thereof are effective for the reduction of body fat.

The present invention further relates to injection compositions of solabegron or its pharmaceutically acceptable salts, esters, solvates, polymorphs, enantiomers, or mixtures thereof and processes for preparing such compositions.

The present invention relates to pharmaceutical compositions (e.g., lyophilized and/or aqueous compositions) comprising solabegron and methods of use thereof.

The present invention also relates to the use of therapeutically effective amounts of injection compositions of solabegron or its pharmaceutically acceptable salts, esters, solvates, polymorphs, enantiomers, or mixtures thereof in the manufacture of a medicament for treating obesity, and its related metabolic disorders, hypertension, patients at risk of having diabetes (pre-diabetes), diabetes, cardiovascular diseases, hyperglycaemia, gallbladder diseases, excess fat on the chin (submental fullness or double chin disorder), binge eating, hypothyroidism, excess fat on the breast, adiposis dolorosa, benign symmetric lipomatosis, lipedema, familial lipodystrophy, familial partial lipodystrophy, HIV lipodystrophy, Bardet-Biedl syndrome, hypertrophy of dorsocervical fat/dorsocervical fat hypertrophy (“buffalo hump”), lipoma, lipomatosis, moon facies, Down syndrome, pseudo-Cushing syndrome, Cohen syndrome, Cushing syndrome, Prader-Willi syndrome, Turner syndrome, or Madelung disease.

### DETAILED DESCRIPTION

The present invention can be more readily understood by reading the following detailed description of the invention and study of the included examples.

As used herein, the term “composition”, “formulation”, “dosage form” as in pharmaceutical composition, is intended to encompass a drug product comprising solabegron or its pharmaceutically acceptable salts, esters, solvates, polymorphs, enantiomers or mixtures thereof, and other inert ingredient(s) (pharmaceutically acceptable excipients). Such pharmaceutical compositions are synonymous with “formulation”, “injectable composition”, “injection composition” and “dosage form” and are used synonymously throughout the application. The term “parenteral” or “injection” as used herein refers to routes selected from subcutaneous (SC), intravenous (IV), intramuscular (IM), intradermal (ID), intraperitoneal (IP), depot injection, and the like. Transcutaneous delivery is also contemplated as a route of delivery for the pharmaceutical compositions as per the present invention. Injection as per the present invention can be injected into the abdomen, chin, waist, arms, legs, knees, thigh, chest, breast, neck, face, buttocks, lateral buttock, peri-orbital region, intra-orbital region, or to a particular fat deposit area. The term “overweight”, as used herein, refers to an adult individual having a body mass index (BMI) greater than or equal to 24 and less than 27. The term “obese”, as used herein, refers to an adult individual having a body mass index (BMI) of greater than or equal to 30.

The compositions as per the present invention include injection preparations, such as liquid dosage forms (liquids, liquid dispersions, solutions, suspensions, emulsions), gels, colloids, dry powder, implants, biodegradable or non-biodegradable microparticles/microspheres in the form of controlled-release formulations, lyophilized formulations, immediate-release formulations, delayed-release formulations, extended-release formulations, sustained-release formulations, pulsatile release formulations, and mixed immediate release and controlled release formulations. Preferably, the composition of the present invention is an immediate release injection composition. An “immediate-release formulation” as used herein, refers to a formulation that has been formulated to allow solabegron to act as quickly as possible. The term “lyophilization” as used herein, refers to freeze-drying or dehydration technique which involves removing a solvent such as water, a water-miscible solvent, from solabegron composition typically by sublimation under high vacuum when the composition is in a frozen state. The term “lyophilized composition”, as used herein refer to solid residue or powder which is produced or which remains after the lyophilization procedure.

As used herein, the terms “β3-adrenergic receptor agonist”, “β3-adrenoreceptor agonists” or “beta-3 adrenergic receptor agonist” include compounds such as solabegron. “Solabegron” is used in a broad sense to include not only solabegron per se (free base) but also its pharmaceutically acceptable salts, solvates, zwitterions, esters, hydrates, enantiomers, derivatives, isomers, stereoisomers, diastereomers, metabolites, polymorphs, and prodrugs thereof. Polymorph may refer to various crystalline and amorphous forms of solabegron.

As used herein, the pharmaceutically acceptable salt(s) include, but are not limited to, maleic, citric, gluconic, lactic, malic, mandelic, cinnamic, citraconic, aspartic, stearic, palmitic, itaconic, glycolic, p-aminobenzoic, glutamic, benzene sulfonic, theophylline acetic acids, fumaric, benzoic, ascorbic, embonic, succinic, oxalic, bismethylenesalicylic, methanesulfonic, ethanedisulfonic, acetic, propionic, tartaric, salicylic, 8-halotheophyllines, hydrochloric, hydrobromic, sulfuric, sulfamic, phosphoric and nitric acids and the like. Other salts such as, but not limited to, amino acids, p-toluenesulfonate, tosylate, dihydrochloride, laurate, hydroiodide, mesylate, benzenesulfonate, bicarbonate, bisulfate, bitartrate, borate, pamoate, palmitate, camsylate, carbonate, methanesulfonate, nitrate, sodium, stearate, are also included. Preferable salts of solabegron include hydrochloride and sodium.

The inventor of the present invention considering the physicochemical properties of solabegron or its various pharmaceutically acceptable salts, esters, prodrugs, solvates, hydrates, polymorphs, to provide an injection composition comprising solabegron, with at least one or more pharmaceutically acceptable excipients, wherein the composition is able to achieve the desired pharmacokinetic profile.

The methods used for the preparation of the injection compositions as per the present invention may be selected from high shear homogenization, high-pressure homogenization, ultrasonication/high-speed homogenization, admixture of solvents, solubilizers and actives to prepare a suspension, solvent emulsification, evaporation and the like.

The term “excipient” means one or more pharmacologically inactive components comprising one or more of solvents, carriers, diluents, bulking agents, emulsifying agents, solubilizing agents, wetting agents, thickening agents, tonicity adjusting agents, pH adjusting agents, viscosity adjusting agents, chelating agents, stabilizers, preservatives, antimicrobial agents, antioxidants, suspending agents, cosolvents, oils, dispersants, polymers, lipids, surface modifiers and combination thereof. Reference to an excipient includes both one and more than one such excipient. Co-processed excipients are also covered under the scope of the present invention. Combination of excipients performing the same function may also be used to achieve desired formulation characteristics. Additional excipients such as oily vehicles and solvents, demulcents, polymeric or hydrophobic materials are also covered under the ambit of the present invention.

The term “patient” and/or “subject” and/or “individual” are used interchangeably herein. In some embodiments, the patient or subject is a human. In further embodiments, the patient or subject is an animal. In some embodiments, the human can be of any age such as adult, adolescent, paediatric or geriatric.

“Disease,” “disorder,” and “condition” are used interchangeably herein. As used herein, the term “therapeutic agent” means an agent utilized to treat, combat, ameliorate, prevent or improve an unwanted condition or allergy, or disease of a patient. “Administering” a composition may be accomplished by parenteral administration and or by other methods alone or in combination with other known techniques. Preferably, the composition according to the present invention is administered parenterally.

A double chin disorder is characterized by extra fat that develops beneath the chin. Benign symmetric lipomatosis (BSL) is a rare proliferative disorder of the adipose tissue, characterized by symmetrical fat deposits, predominantly in the neck and shoulder area, upper back, and arms. Adiposis dolorosa is a rare condition characterized by the growth of multiple, painful, lipomas (benign, fatty tumors). The lipomas may occur anywhere on the body and can cause severe pain. Other symptoms may include weakness, fatigue, and memory disturbances. It usually occurs in adults, and women are more commonly affected than men. Lipedema is a disorder characterized by symmetric enlargement of the legs due to deposits of fat beneath the skin. It is a common condition, occurring almost exclusively in women (affecting up to 11% of women). Familial partial lipodystrophy is a group of rare genetic lipodystrophic syndromes characterized (also known as Köbberling lipodystrophy/Dunnigan lipodystrophy) by fat loss from the limbs and buttocks, from childhood or early adulthood, and often associated with acanthosis nigricans, insulin resistance, diabetes, hypertriglyceridemia, and liver steatosis.

The term “therapeutically effective amount” is such that when administered, the pharmaceutical composition of the present invention provides desired therapeutic effects in the treatment of obesity, fat reduction, and related metabolic disorders and/or other conditions as mentioned in the present invention. The dosage administered to a patient can be as single or multiple doses depending upon a variety of factors, including the drug's administered pharmacokinetic properties, the route of administration, patient conditions and characteristics (sex, age, body weight, health, size, etc.), and extent of symptoms, concurrent treatments, frequency of treatment and the effect desired. The term “non-surgical” refers to a medical procedure that does not require an incision.

The term “instruction” as used herein, includes a publication, a diagram, or any other medium of expression, which can be used to communicate the usefulness and detailed method of administration of the injection composition as per the present invention.

As used in this specification, the singular forms “a”, “an”, and “the” include plural references unless the context clearly dictates otherwise. Thus, for example, a reference to “a process” includes one or more processes, and/or steps of the type described herein and/or which will become apparent to those persons skilled in the art upon reading this disclosure.

“Substantially” as used herein refers to the pharmaceutical composition, which comprises less than 1% or less than 0.5% of alcohol-based preservative including benzyl alcohol and/or glycofurol.

The term “%” or “percent” or “percentage” expressed herein, refers to % w/w or % w/v of the injection composition. Preferably, % w/v of the injection composition.

As used herein, the term “about” means±approximately 10% of the indicated value, such that “about 10 percent” indicates approximately 08 to 12 percent.

The term “stable” refers to the compositions of the present invention, wherein the amount of the active ingredient of a formulation does not deviate from the initial amount by more than the values given in the specification or the guidelines of the common Pharmacopoeias or loss in active ingredient is less than 50% (40%, 30%, 20%, 10%, 5%) of the initial content after being stored for at least 1 month, preferably for at least 2 months, preferably for at least 3 months, more preferably for at least 6 months, more preferably for at least 12 months or more preferably for at least 24 months. The stability of the composition may be evaluated at “long term” conditions 25° C./60% RH, at intermediate condition 30° C./65% RH, at “accelerated conditions” 40° C./75% RH, at refrigerated conditions 2-8° C., in the final container either measured as the loss in the content of active ingredient. Stability testing may be conducted according to the current guidelines by ICH and USFDA. The purity of solabegron in injection compositions as per the present invention ranges from at least 99.99%, 99%, 98%, 97%, 96%, or 95%.

The pharmaceutical compositions of the present invention comprise solabegron or its pharmaceutically acceptable salts, esters, solvates, polymorphs, enantiomers, or mixtures thereof from about 0.05 μg to about 1000 mg per day. In some embodiments, the dose of solabegron or its pharmaceutically acceptable salts, esters, solvates, polymorphs, enantiomers, or mixtures thereof ranges from about 0.05 μg to about 800 mg per day, from about 0.05 μg to about 700 mg per day, about 0.05 μg to about 600 mg per day, about 0.05 μg to about 500 mg per day, about 0.05 μg to about 400 mg per day, about 0.05 μg to about 300 mg per day, about 0.05 μg to about 200 mg per day, about 0.05 μg to about 100 mg per day, about 0.05 μg to about 50 mg per day, about 0.05 μg to about 20 mg per day, about 0.05 μg to about 10 mg per day, about 0.05 μg to about 5 mg per day, about 0.05 μg to about 4.5 mg per day, about 0.05 μg to about 4 mg per day, about 0.05 μg to about 3.5 mg per day, about 0.05 μg to about 3 mg per day, about 0.05 μg to about 2.5 mg per day, about 0.05 μg to about 2 mg per day, about 0.05 μg to about 1.5 mg per day, about 0.05 μg to about 1 mg per day, about 0.05 μg to about 0.5 mg per day.

The pharmaceutical compositions of the present invention comprise solabegron or its pharmaceutically acceptable salts, esters, solvates, polymorphs, enantiomers, or mixtures thereof from about 0.001% and 97% (w/w) or (w/v) active ingredient. In certain embodiments, the composition comprises between about 0.001% to about 90%, between about 0.001% to about 80%, between about 0.001% to about 70%, between about 0.001% to about 60%, between about 0.001% to about 50%, between about 0.001% to about 40%, between about 0.001% to about 30%, between about 0.001% to about 20%, between about 0.001% to about 10%, between about 0.001% to about 5%, or less (w/w) or (w/v), of the active ingredient. In a preferred embodiment, the amount of solabegron or its pharmaceutically acceptable salts, esters, solvates, polymorphs, enantiomers, or mixtures thereof in the composition of the present invention is about 0.001% to about 40% of the total composition.

In an embodiment, administration can occur at least once daily, twice daily, three times a day, four times a day, weekly, once in two weeks, once in three weeks, monthly, once in two months, once in three months, or once in six months.

In an embodiment, the present invention provides pharmaceutical compositions and methods to reduce regional fat, adipose tissue, adipocyte, and regional or localized adiposity.

In an embodiment, the present invention provides a method for treating a condition selected from the group comprising of double chin disorder, benign symmetric lipomatosis, adiposis dolorosa, lipedema, familial partial lipodystrophy by administering a therapeutically effective amount of solabegron or its pharmaceutically acceptable salts, esters, solvates, polymorphs, enantiomers or mixtures thereof.

In an embodiment, the present invention provides a method for reducing or non-surgical removal of body fat in an individual, the method comprising administering to the individual an injection pharmaceutical composition comprising:
a) a compound of the formula:

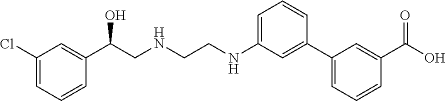 [SMILES: O=C(O)c1cccc(-c2cccc(NCCNC[C@H](O)c3cccc(Cl)c3)c2)c1]

or its pharmaceutically acceptable salts, esters, solvates, polymorphs, enantiomers or mixtures thereof, and
b) at least one or more pharmaceutically acceptable excipients.

In another embodiment, the present invention provides a process of preparing injection compositions comprising solabegron or its pharmaceutically acceptable salts, esters, solvates, polymorphs, enantiomers, or mixtures thereof and at least one or more pharmaceutically acceptable excipients.

In another embodiment, the present invention also relates to injection compositions comprising solabegron or its pharmaceutically acceptable salts, esters, solvates, polymorphs, enantiomers or mixtures thereof and at least one or more pharmaceutically acceptable excipients selected from the group comprising solvent, carrier, diluent, bulking agent, emulsifying agent, solubilizing agent, wetting agent, thickening agent, tonicity adjusting agent, pH adjusting agent, viscosity adjusting agent, chelating agent, stabilizer, preservative, antimicrobial agent, and antioxidant.

In an embodiment, the present invention provides a method for reducing or non-surgical removal of body fat in an individual, the method comprising administering to the individual an injection pharmaceutical composition comprising:
a) a compound of the formula:

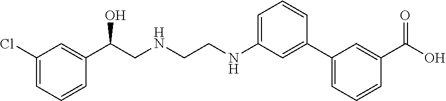 [SMILES: O=C(O)c1cccc(-c2cccc(NCCNC[C@H](O)c3cccc(Cl)c3)c2)c1]

or its pharmaceutically acceptable salts, esters, solvates, polymorphs, enantiomers or mixtures thereof, and
b) at least one or more pharmaceutically acceptable excipients selected from the group comprising solvent, carrier, diluent, solubilizing agent, wetting agent, tonicity adjusting agent, pH adjusting agent, stabilizer, preservative, and antioxidant.

In another embodiment, the pH of the composition may be in the range from pH about 2 to pH about 9.5. Preferably, from pH about 5 to pH about 9. More preferably, the pH is between about 3.0 and about 5.5. The pH may be measured by placing a pH meter directly into the liquid formulation, such as a pH meter having been calibrated for the appropriate pH range with standard aqueous buffers. Persons skilled in the art will know of other methods which may be used to measure pH. These ranges are for measurements made at room temperature (20 to 25° C.). A person skilled in the art will know that the pH meter reading will vary depending on the temperature.

In a further embodiment, the composition has an osmolality that is in the range from 100 to 600 mOsm/kg. In a further embodiment, the composition has an osmolality that is in the range from 100 to 500 mOsm/kg. In a further embodiment, the composition has an osmolality that is in the range from 100 to 400 mOsm/kg. In a further embodiment, the composition has an osmolality equal to and/or less than 350 mOsm/kg.

In another embodiment of the invention, there is provided an injection composition comprising: a) solabegron or its pharmaceutically acceptable salts, esters, solvates, polymorphs, enantiomers, or mixtures thereof, b) a carrier.

In another embodiment of the invention, there is provided an injection pharmaceutical composition comprising lyophilized powder for reconstitution comprising solabegron or its pharmaceutically acceptable salts, esters, solvates, polymorphs, enantiomers or mixtures thereof and at least one or more pharmaceutically acceptable excipients.

In another embodiment of the invention, there is provided an injection composition comprising: a) about 0.01% to about 40% of solabegron or its pharmaceutically acceptable salts, esters, solvates, polymorphs, enantiomers, or mixtures thereof, b) about 20% to about 99% of a pharmaceutically acceptable carrier, and c) at least one or more pharmaceutically acceptable excipients.

In another embodiment of the invention, the injection composition comprising: a) solabegron or its pharmaceutically acceptable salts, esters, solvates, polymorphs, enantiomers, or mixtures thereof, b) a pH adjusting agent, and c) a carrier.

In another embodiment of the invention, the injection composition comprising: a) solabegron or its pharmaceutically acceptable salts, esters, solvates, polymorphs, enantiomers, or mixtures thereof, b) a pH adjusting agent, c) an isotonic agent, and d) a carrier.

In another embodiment of the invention, the injection composition comprising: a) solabegron or its pharmaceutically acceptable salts, esters, solvates, polymorphs, enantiomers or mixtures thereof, b) a preservative, c) a pH adjusting agent, d) an isotonic agent, e) optionally an antioxidant, a chelating agent and a wetting agent, and f) a carrier.

In another embodiment of the invention, the injection composition comprising: a) solabegron or its pharmaceutically acceptable salts, esters, solvates, polymorphs, enantiomers or mixtures thereof, b) a preservative, c) a pH adjusting agent, d) an isotonic agent, e) a chelating agent, f) optionally an antioxidant and a wetting agent, and g) a carrier.

In another embodiment of the invention, the injection composition comprising: a) from about 0.001% to about 80% of solabegron or its pharmaceutically acceptable salts, esters, solvates, polymorphs, enantiomers, or mixtures thereof, b) from 0 to about 30% of preservative, c) from 0 to about 30% of one or more pH adjusting agent, d) from 0 to 40% about of isotonic agent, e) from 0 to 20% about of chelating agent, f) from 0 to 20% about of wetting agent, g) from 0 to 10% about of suspending agent, h) one or more solvents, and optionally, i) one or more other pharmaceutically acceptable excipients.

In another embodiment of the invention, the injection composition of solabegron comprising: a) about 0.01% to about 40% of solabegron or its pharmaceutically acceptable salts, esters, solvates, polymorphs, enantiomers, or mixtures thereof, b) about 0.01% to about 9% of pH adjusting agent, c) about 0.01% to about 10% of a tonicity adjusting agent, and d) about 20% to about 99.99% of a pharmaceutically acceptable carrier.

In another embodiment of the invention, the injection composition of solabegron comprising: a) about 0.01% to about 40% of solabegron or its pharmaceutically acceptable salts, esters, solvates, polymorphs, enantiomers or mixtures thereof, b) about 0.01% to about 7% of preservative, c) about 0.01% to about 9% of pH adjusting agent, d) about 0.01% to about 10% of a tonicity adjusting agent, e) optionally about 0.01% to about 7% of antioxidant, about 0.01% to about 4% of chelating agent and about 0.01% to about 5% of wetting agent, and f) about 20% to about 99.99% of a pharmaceutically acceptable carrier.

In another embodiment of the invention, the injection composition of solabegron comprising: a) about 0.01% to about 40% of solabegron or its pharmaceutically acceptable salts, esters, solvates, polymorphs, enantiomers, or mixtures thereof, b) about 0.01% to about 7% of preservative, c) about 0.01% to about 9% of pH adjusting agent, d) about 0.01% to about 10% of a tonicity adjusting agent, e) about 0.01% to about 4% of chelating agent, f) about 0.01% to about 5% of wetting agent, and g) about 20% to about 99.99% of a pharmaceutically acceptable carrier.

In another embodiment of the invention, the injection composition of solabegron comprising: a) about 0.01% to about 40% of solabegron or its pharmaceutically acceptable salts, esters, solvates, polymorphs, enantiomers, or mixtures thereof, b) about 0.01% to about 8% of pH adjusting agent, c) about 0.01% to about 10% of a tonicity adjusting agent, d) about 0.01% to about 5% of wetting agent, and e) about 20% to about 99.99% of a pharmaceutically acceptable carrier.

In another embodiment of the invention, there is provided a liquid pharmaceutical composition suitable for parenteral administration comprising: a) about 0.001% to about 10% of solabegron, b) about 0.01% to about 5% of one or more pH adjusting agents, c) from 0.001% to about 1% of one or more wetting agents, d) from 0.001% to about 2% of one or more tonicity adjusting agents, and e) one or more parenteral solvents.

In another embodiment of the invention, the solabegron injection composition is substantially free or free from preservative and/or antioxidant.

In another embodiment of the invention, the solabegron injection composition is substantially free or free from alcohol-based preservative. In another embodiment of the invention, the injection composition of solabegron is substantially free from benzyl alcohol. In another embodiment of the invention, the injection composition of solabegron is free from benzyl alcohol.

In another embodiment of the invention, the solabegron injection composition is substantially free or free from glycofurol.

In another embodiment of the invention, the injection composition of solabegron or its pharmaceutically acceptable salts, esters, solvates, polymorphs, enantiomers, or mixtures thereof is lyophilized to provide for a stable composition which is ready to be reconstituted by the addition of the appropriate amount of water.

In another embodiment of the invention, the present invention provides a lyophilized powder for reconstitution for parenteral administration comprising: a) solabegron or its pharmaceutically acceptable salts, esters, solvates, polymorphs, enantiomers or mixtures thereof, b) a preservative, c) a pH adjusting agent, d) a tonicity adjusting agent, and e) optionally an antioxidant, a chelating agent and a wetting agent.

In another embodiment of the invention, there is provided a liquid pharmaceutical composition suitable for parenteral administration comprising:
a) about 0.001% to about 90% of solabegron,
b) about 0.01% to about 70% of one or more pH adjusting agents, and
c) one or more parenteral solvents.

In another embodiment the injection composition of solabegron or its pharmaceutically acceptable salts, esters, solvates, polymorphs, enantiomers or mixtures thereof includes particle size of solabegron, having a particle size distribution such that D₉₀ is less than about 200 μm, D₅₀ is less than about 100 μm and D₁₀ is less than about 50 μm. Preferably, particle size distribution is D₉₀ is less than about 100 μm, D₅₀ is less than about 70 μm and D₁₀ is less than about 30 μm. The terms D₁₀, D₅₀, and D₉₀ used herein as per the present invention indicate that 10%, 50%, and 90% of the distribution is below this value. The particle size of solabegron can be achieved by any well-known particle size reduction processes, such as sifting, milling, micronization, fluid energy milling, media milling, ball milling, milled through high-pressure homogenizer, air-jet milling, and the like. The particle size of solabegron can be measured by suitable techniques such as Laser light scattering (e.g. Malvern Light Scattering), Coulter counter, microscopy, and any other technique known in the art.

In another embodiment of the invention, the weight ratio of solabegron to the said wetting agent is 10:0.001 to 0.001:10. Preferably, the weight ratio of solabegron to the said wetting agent is 10:0.01 to 0.01:10. Preferably, the weight ratio of solabegron to the said wetting agent is 10:1.

In another embodiment of the invention, the weight ratio of solabegron to the said tonicity adjusting agent is 10:0.001 to 0.001:10. Preferably, the weight ratio of solabegron to the said tonicity adjusting agents is 10:0.01 to 0.01:10.

In another embodiment of the invention, the pH adjusting agent is present in the composition in a concentration of about 0.001 mg/ml to about 50 mg/ml. Preferably, from about 0.01 mg/ml to about 20 mg/ml.

In another embodiment, the injection composition has a viscosity of about 0.5 poise to about 50 poise at a shear rate of 1/s at 25° C. In some embodiments, the composition has a viscosity of about 0.5 poise to about 10 poise at a shear rate of 10/s at 25° C. In some embodiments, the composition has a viscosity of about 0.5 poise to about 4 poise at 100/s shear rate at 25° C.

In another embodiment, the injection composition of solabegron or its pharmaceutically acceptable salts, esters, solvates, polymorphs, enantiomers, or mixtures thereof includes sterilization. Various sterilization procedures include, but are not limited to, heat sterilization, terminal steam sterilization, dry heat sterilization, moist heat sterilization, filtration, membrane sterilization, radiation or gamma sterilization, and the like. After the sterilization, the injection composition is aseptically packed into a respective container. In alternate embodiments, the composition is terminally sterilized or prepared in strict sterile conditions. The composition can be prepared using sterile water or water for injection (WFI).

In another embodiment, the composition comprises less than 3 percent of impurities, preferably less than 1 percent, more preferably less than 0.5 percent of impurities.

In another embodiment, the composition is free of microbial content during storage.

In another embodiment, the injection composition of solabegron or its pharmaceutically acceptable salts, esters, solvates, polymorphs, enantiomers, or mixtures thereof is stable for at least 3 months, preferably for 6 months, more preferably for 12 months, and more preferably for 24 months when stored at room temperature or refrigerated conditions.

In another embodiment, the injection composition of solabegron or its pharmaceutically acceptable salts, esters, solvates, polymorphs, enantiomers, or mixtures thereof is free from precipitation during shelf-life. Precipitation or homogeneity can be measured using various known techniques such as using light scattering or observing visual clarity of composition. In another embodiment, the injection composition as per the present invention has a relative standard deviation for homogeneity of less than about 4%.

In another embodiment, the injection composition has the advantages of low dosage, high stability, high-fat tissue bioavailability, and few side effects.

In another embodiment, the injection composition of solabegron or its pharmaceutically acceptable salts, esters, solvates, polymorphs, enantiomers, or mixtures thereof remains stable and does not degrade during sterilization conditions in a stoppered vial under nitrogen by heating with steam at 121° C. for at least 15 minutes.

In another embodiment, the injection composition of solabegron or its pharmaceutically acceptable salts, esters, solvates, polymorphs, enantiomers, or mixtures thereof is expected to exhibit desired formulation technical attributes such as particle size, clarity, ease of manufacturing, drug sterility, pH, viscosity, drug release, dosage regimen, stability, patient compliance, and commercial viability.

In further embodiments, the injection composition of solabegron or its pharmaceutically acceptable salts, esters, solvates, polymorphs, enantiomers, or mixtures thereof is expected to exhibit desired pharmaceutical technical attributes in the following tests: such as foreign and particulate matter test, sterility test, bacterial endotoxin test and package integrity leak test for container-closure integrity.

In another embodiment of the invention, the injection composition is prepared by conventional methods for preparation in the art, including but not limited to, mixing, freeze-drying, spray-drying method, solvent-volatilizing method, emulsion-solvent volatilization, sterile filtration, recrystallization followed by aseptic micronization, dry or wet milling followed by gamma or e-beam irradiation sterilization and atomizing-extracting method. Other formulation techniques are also contemplated within the scope of the present invention.

In another embodiment of the invention, the injection composition is prepared by dissolving or suspending the solabegron or its pharmaceutically acceptable salts, esters, solvates, polymorphs, enantiomers, or mixtures thereof in a pharmaceutically acceptable vehicle under sterile conditions.

In another embodiment of the invention, the process of preparing the injection composition is carried out under aseptic conditions, and when grinding, the temperature should not exceed 40° C.

In one embodiment, the invention relates to a process for preparing an injection pharmaceutical composition of solabegron or its pharmaceutically acceptable salts, esters, solvates, polymorphs, enantiomers, or mixtures thereof comprising the steps of: a) preparing a mixture comprising solabegron or its pharmaceutically acceptable salts, esters, solvates, polymorphs, enantiomers or mixtures thereof, carrier and one or more other pharmaceutically acceptable excipients.

In one embodiment, the invention relates to a process for preparing an injection composition comprising: a) preparing a first sterile solution comprising one or more pharmaceutically acceptable excipients, b) preparing a second sterile solution comprising solabegron or its pharmaceutically acceptable salts, esters, solvates, polymorphs, enantiomers or mixtures thereof, and c) dispersing both solutions to form the final sterile composition.

In one embodiment, the invention relates to a process for preparing an injection composition comprising: a) dissolving the drug in a solvent, b) charging the drug solution in another suitable solvent, cooling below an appropriate temperature, and c) filtering the particles of the drug, thus obtaining sterile particles.

In one embodiment, the invention relates to a process for preparing an injection pharmaceutical composition of solabegron or its pharmaceutically acceptable salts, esters, solvates, polymorphs, enantiomers, or mixtures thereof comprising one or more additional steps selected from the group consisting of a) purging the mixture with an inert gas, and/or b) filtering the mixture through a filter e.g. with a filter having an average pore size of not more than 0.5 μm and/or, c) filling the mixture into a suitable container, and d) autoclaving the mixture at elevated temperature and elevated pressure e.g. at 121° C. and 2 bar for at least 20 minutes.

The injection compositions of the present invention are expected to exhibit desired technical characteristics based on tests such as a) pH: pH is measured by using a pH meter, 2) Sterility Test: It can be carried out by inoculation of a culture medium with the composition. If there is no evidence of microbial growth then the preparation being examined passes the test for sterility, 3) Leakage test: Leakage test is employed to test the package integrity. Leakage test can be done by dye bath test. The test container is immersed in a dye bath. Vacuum and pressure are applied for some time. The container is removed from the dye bath and washed. The container is then inspected for the presence of dye either visually or by means of UV spectroscopy, 4) Pyrogen test, 5) Content uniformity, 6) Viscosity, 7) Clarity, 8). Drug release and 9) Stability.

In one embodiment, the present invention provides a method of fat reduction by reducing the number of fat cells, the volume of fat cells as measured by at least one of volume, size, mass, bulk, density, amount, and/or quantity. The present invention is expected to reduce fat by greater than or equal to 80%, greater than or equal to 60%, greater than or equal to 50%, greater than or equal to 30%, greater than or equal to 20%, greater than or equal to 10%, or greater than or equal to 5%.

In one embodiment, the present invention provides a method of administering an injection composition intramuscularly or subcutaneously as described in the present invention.

In an embodiment, the composition as per the present invention is packed in a suitable container selected from a vial, ampoule, syringe, pen (single or multi-compartment), auto-injector, cartridge.

In one embodiment, the present invention provides a kit comprising: a) an injection composition of solabegron or its pharmaceutically acceptable salts, esters, solvates, polymorphs, enantiomers, or mixtures thereof in a suitable container like a vial, ampoule, syringe, auto-injector, pen (single or multi-compartment) and optionally, b) a container comprising a diluent or a solvent or a carrier for preparing the composition, c) instructions for preparing and administration of the composition for reducing fat deposit in a subject without the use of surgery.

In another embodiment, the injection composition of solabegron or its pharmaceutically acceptable salts, esters, solvates, polymorphs, enantiomers, or mixtures thereof can be administered as a single dose or multiple doses such that the patients receive repeated doses over several hours, days, weeks, months, or more. The multiple dosing method may comprise first dose administration followed by second dose administration. The second dose can be the same, or different from the first dose.

In one embodiment, the present invention provides a pharmaceutical kit, wherein the composition comprising a drug and one or more excipients contained in a first container, and the suitable solvent or diluent is contained in a second, separate container. Preferably, at least one of the first and second containers is a syringe, auto-injector, an ampoule, a vial, a pen, or a cartridge, either disposable or not, which can be single-use or multiple uses. In one aspect of the present invention, the composition is directed to a kit comprising a first container selected from a syringe, auto-injector, vial, ampoule, pen, or cartridge, containing at least one excipient and a drug in the appropriate amounts and a second container selected from a syringe, vial, ampoule, auto-injector, pen or cartridge comprising at least one excipient like solvents or diluents. When required, the contents of both containers are combined, for example through a connector or by using male-female syringes and mixed so that the compositions according to the invention are reconstituted, for example by moving forwards and backward the plungers of the syringes. The container used as per the present invention can be sealed airtight, allowing no gas exchange from the outside environment and keep the contents of the composition sterile. In an alternate embodiment, the container does not allow oxygen ingress and oxygen content is maintained at a minimal level to avoid oxidative degradation.

In certain non-limiting embodiments, the container includes, but is not limited to, glass vials (for example, but not limited to, flint glass vials), ampoules, plastic flexible containers, for example, but not limited to, PVC (polyvinyl chloride) containers, VisIV™ plastic containers, CR3 elastomer copolyester ether containers, CZ resin containers, polypropylene containers, and syringes.

The prefilled syringes or vials or ampoules or pens or cartridges or auto-injector as per present invention may contain volumes from about 10 ml or less, 9.5 ml or less, 9 ml or less, 8.5 ml or less, 8 ml or less, 7.5 ml or less, 7 ml or less, 6.5 ml or less, 6 ml or less, 5.5 ml or less, 5 ml or less, 4.5 ml or less, 4 ml or less, 3.5 ml or less, 3 ml or less, 2.5 ml or less, 2 ml or less, 1.5 ml or less, 1 ml or less, 0.5 ml or less, 0.1 ml or less.

In another embodiment of the invention, there is provided a use of therapeutic effective amount of injection composition of solabegron or its pharmaceutically acceptable salts, esters, solvates, polymorphs, enantiomers or mixtures thereof in the manufacture of medicament for treating obesity, and its related metabolic disorders, hypertension, patients at risk of having diabetes (pre-diabetes), diabetes, cardiovascular diseases, hyperglycaemia, gallbladder diseases, excess fat on the chin (submental fullness or double chin disorder), binge eating, hypothyroidism, excess fat on the breast, adiposis dolorosa, benign symmetric lipomatosis, lipedema, familial lipodystrophy, familial partial lipodystrophy, HIV lipodystrophy, Bardet-Biedl syndrome, hypertrophy of dorsocervical fat/dorsocervical fat hypertrophy (“buffalo hump”), lipoma, lipomatosis, moon facies, Down syndrome, pseudo-Cushing syndrome, Cohen syndrome, Cushing syndrome, Prader-Willi syndrome, Turner syndrome, or Madelung disease.

In another embodiment of the invention, there is provided a use of the therapeutic effective amount of injection composition of solabegron or its pharmaceutically acceptable salts, esters, solvates, polymorphs, enantiomers, or mixtures thereof for treating a condition selected from the group comprising of double chin disorder, benign symmetric lipomatosis, adiposis dolorosa, lipedema, familial partial lipodystrophy.

In an embodiment, the injection composition of solabegron or its pharmaceutically acceptable salts, esters, solvates, polymorphs, enantiomers, or mixtures thereof is administered into adipose tissue. In an embodiment, the injection composition locally reduces adipose tissue. In a further embodiment, the injection composition reduces submental adipose tissue. In an embodiment, the fat is reduced from a body part selected from the group consisting of the abdomen, chin, waist, arm, leg, knee, thigh, chest, breast, neck, face, buttock, lateral buttock, peri-orbital region, and intra-orbital region.

In another embodiment of the invention, there is provided a use of a therapeutic effective amount of injection composition of solabegron or its pharmaceutically acceptable salts, esters, solvates, polymorphs, enantiomers, or mixtures thereof for the reduction of submental fat (double chin) in a subject, said method comprising administering about 0.001 mg to about 40 mg of said solabegron or its pharmaceutically acceptable salts, esters, solvates, polymorphs, enantiomers or mixtures thereof per square centimeter of the skin area over said submental fat.

In another embodiment of the invention, the pharmaceutical composition as per the present invention is administered within a plurality of treatment sessions. In a further embodiment, each treatment session is spaced by at least 1 day. In other embodiments, each treatment session is separated from another treatment session by at least 2 to 30 days. In another embodiment of the invention, a plurality of treatment sessions may include administration of injection composition up to a maximum of 80 injections.

In another embodiment of the invention, a plurality of treatment sessions by injection composition are spaced at least about 0.1 cm apart. In further embodiments, the plurality of treatment sessions by injection composition are spaced from about 0.1 cm to about 10 cm apart. In another embodiment, the plurality of treatment sessions by injection composition are spaced about 0.3 cm apart. In a further embodiment, the space is usually measured by a marker to be applied to the affected area of the patient.

In another embodiment of the invention, the patient has excess body fat as a side effect of medication, due to changes in hormonal status, smoking cessation, age-related, and/or due to any surgery.

Experimental tests for the effect of solabegron or its pharmaceutically acceptable salts, esters, solvates, polymorphs, enantiomers, or mixtures thereof in reducing fat deposition in a subject in need thereof include, Pre-clinical studies including: 1) In-vitro tests (In Vitro Study of Mouse Preadipocyte Viability, Preadipocyte Differentiation, Preadipocyte Apoptosis, and Adipocyte Apoptosis), 2) Animal study (effect of solabegron on inguinal lateral fat pad of hamsters/rats), 3) Clinical studies including: i) In vivo human study, ii) Body mass index (BMI) study (Quantitative methods for the analysis of weight loss or maintenance include measurements of body mass index (BMI). In some embodiments, BMI may be monitored by determining a subject's body mass and height and then dividing the individual's body mass by the square of their height, with the value given in units of kg/m. BMI values may range from underweight to obesity and may be used to assess how much a subject's body weight departs from what is normal or desirable for a person of his or her height.

The solvents or carriers or diluents used in the present invention are non-toxic, biocompatible, and appropriate for injection dosage forms. More preferably, selected solvents are biocompatible in order not to cause any severe tissue irritation or necrosis at the injection site. Various useful solvent(s) include, but are not limited to, aqueous, non-aqueous solvents, oils, C₂-C₆ aliphatic alcohols, ethanol, 1-propanol, 2-propanol, 1-butanol, 2-butanol, 1-pentanol, 2-pentanol, 3-pentanol, isopropanol, benzyl alcohol, glycol ethers (e.g., including, but limited to, diethyleneglycol monoethyl ether (DGME, Transcutol®)), butyl diglycol, dipropylene glycol n-butyl ether, ethyleneglycol monoethyl ether, dipropylene glycol monomethyl ether, propylene glycol monomethyl ether, propylene glycol monoethyl ether, liquid polyethylene glycols (PEGs) (PEG 200, PEG 300, PEG 400), carbonates (e.g., propylene carbonate), 2-pyrrolidone, N-methylpyrrolidone, dimethyl isosorbide, dimethylacetamide, glycerol formal, dichloromethane, chloroform, ethyl acetate, dioxane, ethyl ether, acetone, tetrahydrofuran, benzene, toluene, glacial acetic acid, petroleum ether, alkane, paraffine, dimethylsulfoxide, liquid polyethylene glycol, block copolymers of oxyethylene, polyoxyethylene alcohol, polyoxyethylene fatty acid esters, hydrocarbons, n-propane, n-butane, isobutane, n-pentane, iso-pentane, neo-pentane, n-hexane, ethers, diethyl ether, hydroxylated solvents, dextrose, aqueous saline, water, purified water, diethylene glycol ethyl ether, isopropylidene glycerol, glycerol, N-methyl-pyrrolidone, N-pyrrolidone, methylethylketone, 1-dodecylazacycloheptane, dipropyleneglycol methyl ether, methyl acetate, ethyl lactate, dimethylformamide, N,N-diethyl-m-toluamide, ethylacetamide, caprolactam, decylmethylsulfoxide, triacetin and mixtures thereof. In an embodiment, the solvent according to the present invention is present in an amount of about 99.99% or less, e.g. 90% or less, 80% or less, 70% or less, 60% or less, 50% or less, 40% or less, 30% or less, 20% or less, 10% or less.

Various useful solubilizing agents, wetting agents include, but are not limited to, beta-cyclodextrin, sodium lauryl sulfate, sorbitan monolaurate, sorbitan tristearate, sorbitan monooleate, sodium oleate, diethylene glycol monostearate, diethylene glycol monolaurate, glyceryl monostearate, polyoxyethylene sorbitol beeswax, polyethylene lauryl ether, polyoxyethylene lauryl ether, polyoxyethylene monostearate, polyoxyethylene alkyl phenol, polyethylene sorbitan monooleate, polyethylene sorbitan monolaurate, polyoxyethylene lauryl ether, potassium oleate, triethanolamine oleate or mixtures thereof. Solubilizing agents or wetting agents as per the present invention can also be used as amphiphilic agents. In an embodiment, the solubilizing agent, wetting agent according to the present invention is present in an amount of about 50% or less, e.g. 40% or less, 30% or less, 20% or less, 10% or less, 5% or less.

Various thickening agent(s) include, but are not limited to, hydroxypropylmethylcellulose, hydroxypropylcellulose, methylcellulose, hydroxyethylcellulose, carboxymethylcellulose, carbomer, polyvinylpyrrolidone, polyvinyl alcohol, natural gums, methyl cellulose, xanthan gum, carboxymethyl cellulose, carbomer, alginates, acacia, chitosans, gelatin, tragacanth, ethoxose, alginic acid, polyacrylic acid, pectin, alone or in combination thereof. In an embodiment, the thickening agent according to the present invention is present in an amount of about 20% or less, e.g. 10% or less, 5% or less, 2.5% or less, or 2% or less.

Various diluent(s) or carrier(s) include, but are not limited to, mannitol, glycine, lactose, sucrose, trehalose, dextran, hydroxyethyl starch, ficoll or gelatin, and the like. The amount of diluents may range from about 0.01% to about 90% of the composition. In an embodiment, the diluent according to the present invention is present in an amount of about 90% or less, e.g. 80% or less, 70% or less, 60% or less, 50% or less, 40% or less, 30% or less, 20% or less, 10% or less, 5% or less, 2.5% or less, 2% or less.

Various useful tonicity adjusting agent(s) include, but are not limited to, potassium chloride, mannitol, glycerin, lactose, glycerol, dextrose, sodium chloride, sodium sulfate, sorbitol, trehalose, ammonium carbonate, ammonium chloride, ammonium lactate, ammonium nitrate, ammonium phosphate, ammonium sulfate, ascorbic acid, bismuth sodium tartrate, boric acid, calcium chloride, disodium calcium edetate, calcium gluconate, calcium lactate, citric acid, diethanolamine, dimethyl sulfoxide, disodium edetate, trisodium edetate monohydrate, sodium fluorescein, fructose, galactose, glycerin, lactic acid, lactose, magnesium chloride, magnesium sulfate, polyethylene glycol, potassium acetate, potassium chlorate, potassium chloride, potassium iodide, potassium nitrate, potassium phosphate, potassium sulfate, propylene glycol, silver acid, sodium acetate, sodium bicarbonate, sodium biphosphate, sodium bisulfite, sodium borate, sodium bromide, sodium cacodylate, sodium carbonate, sodium citrate, sodium iodide, sodium lactate, metabisulfate sodium sulfite, sodium nitrate, sodium nitrite, sodium phosphate, sodium propionate, sodium succinate, sodium sulfite, sodium tartrate, sodium thiosulfate, maltose, sucrose, tartaric acid, triethanolamine, urea, urethane, uridine zinc sulfate, zinc chloride, albumin, amino acid alone or in combination thereof. In an embodiment, the tonicity adjusting agent according to the present invention is present in an amount of about 60% or less, e.g. 50% or less, 40% or less, 30% or less, 20% or less, 10% or less, 5% or less.

Various useful pH stabilizer(s) or pH adjusting agent(s) or buffer(s) include, but are not limited to, inorganic acids, organic acids, inorganic bases, and organic bases, acetic acid/acetate, sodium acetate, ascorbic acid, sodium ascorbate, sodium ethoxide, gluconate buffer, sodium carbonate, sodium hydroxide, potassium hydroxide, disodium hydrogen phosphate anhydrous, sodium dihydrogen phosphate monohydrate, magnesium carbonate, calcium carbonate, magnesium oxide, ammonia, hydrochloric acid, malic acid/malate, citric acid/citrate, sulfuric acid, nitric acid, phosphoric acid/phosphate, adipic acid, benzoic acid, sodium benzoate, boric acid, potassium phosphate, monobasic sodium acetate, sodium bicarbonate, tris buffer, sodium borate, glycine/glycimate, maleic acid, monobasic sodium phosphate, sodium diphosphate, HEPES, lactic acid/lactate, tartaric acid/tartrate, fumaric acid, potassium metaphosphate, sodium tartrate, anhydrous sodium citrate, dihydrate and combination thereof. Other buffering agents also include citric acid/phosphate mixture, acetate, barbital, Britton-robinson, cacodylate, collidine, formate, maleate, mclvaine, glutamic acid/glutamate, prideaux-ward, succinate, citrate-phosphate-borate (Teorell-Stanhagen), veronal acetate, MES (2-(N-morpholino) ethanesulfonic acid), Bis-Tris (bis (2-Hydroxyethyl) imino-tris (hydroxymethyl) methane), ADA (N-(2-acetamido)-2-iminodiacetic acid), ACES (N-(carbamoylmethyl)-2-aminoethanesulfonic acid), PIPES (piperazine-N, N′-bis (2-ethanesulfonic acid)), MOPSO (3-(N-Morpholino)-2-hydroxypropanesulfonic acid), bistris propane (1,3-bis (tris (hydroxymethyl) methylamino) propane), BES (N, N-bis (2-hydroxyethyl)-2-aminoethane Sulfonic acid), MOPS (3-(N-morpholino) propanesulfonic acid), TES (N-tris (hydroxymethyl) methyl-2-aminoethanesulfonic acid), HEPES (N-(2-hydroxyethyl) piperazine-N′-(2-ethanesulfonic acid), dipso (3-(N, N-bis (2-hydroxyethyl) amino)-2-hydroxypropanesulfonic acid), MOBS (4-(N-morpholino)-butanesulfonic acid), tricine (N-tris (hydroxymethyl)) Methylglycine), GLY-GLY (glycylglycine), bicine (N, N-bis (2-hydroxyethyl) glycine), HEPBS (N-(2-hydroxyethyl) piperazine-N′-(4-butanesulfone) Acid)), TAPS (N-tris (hydroxymethyl) methyl)-3-amino-propanesulfonic acid), AMPD (2-amino-2-methyl-1,3-propanediol), Tapso (3-(N-tris (hydroxymethyl) methylamino)-2-hydroxy Propanesulfonic acid), Trizma™ (Tris (hydroxymethylaminomethane), Heppso (N-(2-hydroxyethyl) piperazine-N′-(2-hydroxypropanesulfonic acid), popso (piperazine-N, N′)-Bis (2-hydroxypropanesulfonic acid)), TEA (triethanolamine), EPPS (N-(2-hydroxyethyl) piperazine-N′-(3-propanesulfonic acid), alone or in combination thereof. In an embodiment, the pH stabilizer according to the present invention is present in an amount of about 40% or less, e.g. 30% or less, 20% or less, 10% or less, 5% or less.

Suitable viscosity adjusting agents or suspending agents are selected from the group comprising povidone, polyvinylpyrrolidone compounds, and polyethylene glycols, cellulose derivatives e.g. methylcellulose, carboxymethylcellulose, sodium carboxymethylcellulose, hydroxyethylcellulose and hydroxypropylmethylcellulose, and combinations thereof. The viscosity adjusting agent or suspending agent is present in an amount ranging from about 0.1% to about 10%, particularly from about 0.5% to about 5%.

Various useful preservative(s) include, but are not limited to, ethanol, parabens (methylparaben and/or propylparaben), benzalkonium chloride, benzethonium chloride, methyl, ethyl, propyl, and butyl esters of hydrobenzoic acid, benzoic acid, imidura, benzyl alcohol, bronopol, butylparaben, cetrimide, chlorhexidine, chlorobutanol, chlorocresol, cresol, ethylparaben, imidurea, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, m-cresol, phenol, phenylmercuric salts, butylated hydroxyltoluene (BHT), butylated hydroxyanisole (BHA), propyl gallate, tocopherols, DMDM Hydantoin®, Euxyl® K400, Bronopol® (2-bromo-2-nitropropane-1,3-diol), chlorhexidine, 2-phenoxyethanol, chlorbutol, thiomersal and the like and mixtures thereof. In an embodiment, the preservative according to the present invention is present in an amount of about 40% or less, e.g. 30% or less, 20% or less, 10% or less, 5% or less, 2.5% or less.

Various useful chelating agents include, but are not limited to, ethylenediamine tetraacetic acid salts (e.g. edetate disodium), diethylenetriaminepentaacetic acid (DTPA), ethylene glycol-bis(β-aminoethyl ether)-tetraacetic acid (EGTA), N-(hydroxy ethyl) ethylenediaminetriacetic acid (HEDTA), 8-hydroxyquinoline, citric acid, tartaric acid, phosphoric acid, gluconic acid, and combinations thereof. The chelating agent is present in an amount ranging from about 0.001% to about 30%.

Various useful antioxidant(s) include, but are not limited to, ascorbic acid and its salts, butylated hydroxytoluene, butylated hydroxyanisole, metal chelators such as ethylenediaminetetraacetic acid, ascorbyl palmitate, benzoic acid, benzyl alcohol, tocopherol, vitamin E, alpha-tocopherol, ascorbyl palmitate, sodium metabisulfite, sodium bisulphite, propyl gallate, n-propyl gallate, methionine, fumaric acid, malic acid, sodium ascorbate, BHA (butylated hydroxy anisole), citric acid, monothioglycerol, tert-butyl hydroquinone (TBHQ), phenols, myristyl-gamma-piccolinium chloride, phenylmer curic acetate and the like and mixtures thereof. In an embodiment, the antioxidant according to the present invention is present in an amount of about 40% or less, e.g. 30% or less, 20% or less, 10% or less, 5% or less, 2.5%, or less.

Various useful stabilizer(s) include, but are not limited to, one or more of EDTA (ethylene diamine tetraacetic acid), para-hydroxybenzoic acid ester derivatives, alcohol, benzalkonium chloride, phenol derivatives, thiomersal, acetic anhydride, ascorbic acid, sorbic acid, boric acid, adipic acid, sodium carboxylate, lauryl sulfate, retinol, tocopherol or sodium ascorbate, sulfite compounds, amino acid such as L-cysteine, thiodipropionic acid, thiolactic acid, and monothioglycerol, sulfurous acid, sulfite, ascorbate, L-cysteine, and tocopherol. N-acetyl amino acid, tocopherol, sodium formaldehyde, or tertiary-butyl hydroquinone, sodium sulfite, sodium phosphate, and the like. In an embodiment, the stabilizer according to the present invention is present in an amount of about 30% or less, e.g. 20% or less, 10% or less, 5% or less, 2.5% or less.

Various useful emulsifying agent(s) or amphiphilic agent(s) include, but are not limited to ethanol, Vitamin E TPGS, soybean phospholipid, egg yolk lecithin, dimyristoyl phosphatidyl choline, dipalmitoyl phosphatidyl choline, distearoyl phosphatidylcholine, dioleoyl phospholipid phatidylcholine, palmityl oleoyl phosphatidylcholine, DSPE, poloxamer, polyoxyethylene sorbitan fatty esters or polysorbates, including, polysorbate 80, polysorbate 20, polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate, tween, span, lecithins, alginic acid, sodium alginate, potassium alginate, ammonium alginate, calcium alginate, propane-1,2-diol alginate, agar, carrageenan, locust bean gum, guar gum, tragacanth, acacia, xanthan gum, karaya gum, pectin, amidated pectin, ammonium phosphatides, microcrystalline cellulose, methylcellulose, hydroxypropylcellulose, hydroxypropylmethylcellulose, ethylmethylcellulose, carboxymethylcellulose sodium, potassium and calcium salts of fatty acids, mono- and di-glycerides of fatty acids, acetic acid esters of mono- and di-glycerides of fatty acids, lactic acid esters of mono- and di-glycerides of fatty acids, citric acid esters of mono- and di-glycerides of fatty acids, tartaric acid esters of mono- and di-glycerides of fatty acids, mono- and diacetyltartaric acid esters of mono- and di-glycerides of fatty acids, mixed acetic and tartaric acid esters of mono- and di-glycerides of fatty acids, sucrose esters of fatty acids, sucroglycerides, polyglycerol esters of fatty acids, polyglycerol esters of polycondensed fatty acids of castor oil, propane-1,2-diol esters of fatty acids, sodium stearoyl-2lactylate, calcium stearoyl-2-lactylate, stearoyl tartrate, extract of quillaia benzyl alcohol, N-methyl-2-pyrrolidone, dimethylacetamide, polyglycerol esters of dimerised fatty acids of soya bean oil, oxidatively polymerised soya bean oil, pectin extract, alone or in combination thereof. Emulsifying agents as per the present invention can also be used as solubilizing agents or wetting agents. In an embodiment, the emulsifying agent according to the present invention is present in an amount of about 40% or less, e.g. 30% or less, 20% or less, 10% or less, 5% or less.

Various useful wetting agent(s) include, but are not limited to, one or more of anionic, cationic, non-ionic, or zwitterionic surfactants or mixtures thereof such as sodium lauryl sulphate, polysorbate (e.g. polysorbate 20, polysorbate 40, polysorbate 60, and polysorbate 80), cetrimide, cetyl alcohol, stearyl alcohol, cetyl stearyl alcohol, cholesterol, polyethylene glycols, polyglycerin fatty acid esters such as decaglyceryl monolaurate and decaglyceryl monomyristate, sorbitan fatty acid esters such as sorbitan monostearate, polyoxyethylene sorbitan fatty acid ester such as polyoxyethylene sorbitan monooleate, polyoxyethylene alkyl ether such as polyoxyethylene lauryl ether, polyoxyethylene castor oil, polyoxyethylenepolyoxypropylene block copolymers such as poloxamer (such as poloxamer 124, poloxamer 188, poloxamer 237, poloxamer 338, and poloxamer 407), and combinations thereof. The amount of surfactant according to the present invention ranges from about 0 to about 50% by weight of the composition. In an embodiment, the wetting agent according to the present invention is present in an amount of about 50% or less, e.g. 40% or less, 30% or less, 20% or less, 10% or less, 5% or less, or 2% or less.

The invention is further defined by reference to the following examples described in detail below. It will be apparent to those skilled in the art that many modifications, both to materials and methods, may be practiced without departing from the scope of the invention.

The following solabegron injection compositions are expected to exhibit the desired technical attributes in the treatment of reducing body fat as given in Table 1 and 2, under sterile conditions as per processes as mentioned in the present invention, and filled into a suitable container.

TABLE 1

| | Quantity (%) | | | |
|---|---|---|---|---|
| Ingredients | Example 1 | Example 2 | Example 3 | Example 4 |
| Solabegron | 0.01-80 | 0.01-40 | 0.01-40 | 0.01-30 |
| Sodium phosphate | 0-10 | 0.1-6 | 0.1-6 | 0.1-3 |
| Sodium chloride | 0-10 | 0.01-3 | 0.01-3 | 0.01-1 |
| Polysorbate 80 | 0-10 | — | — | — |
| Disodium edetate | 0-5 | — | — | — |
| Sodium Hydroxide | 0-5 | 0.01-3 | 0.01-3 | 0.01-1 |
| Benzyl Alcohol | 0-0.9 | 0.01-0.5 | — | — |
| Water | q.s. | q.s. | q.s. | q.s. |

TABLE 2

| | Quantity (%) w/w | | | | | |
|---|---|---|---|---|---|---|
| Ingredients | 5 | 6 | 7 | 8 | 9 | 10 |
| Solabegron | 1 | 1 | 5 | 1 | 10 | 5 |
| Sodium acetate | — | — | 0.5 | 0.5 | 0.5 | 0.5 |
| Sodium phosphate monobasic | — | 0.2 | — | — | — | — |
| Sodium phosphate dibasic | — | 0.5 | — | — | — | — |
| Citric acid | 0.2 | — | — | — | — | — |
| Sodium citrate | 0.2 | — | — | — | — | — |
| Polysorbate-80 | — | — | — | — | — | 0.5 |
| Glacial acetic acid | — | — | q.s. | q.s. | q.s. | q.s. |
| Hydrochloric acid | q.s. | q.s. | — | — | — | — |
| Water For Injection | q.s. | q.s. | q.s. | q.s. | q.s. | q.s. |

Procedure: a) collect a suitable vehicle (such as water for injection) in a suitable container; b) cool the vehicle of step a) to a suitable temperature range (such as 20 to 25° (C.); c) add suitable excipients (such as a wetting agent, tonicity adjusting agent) with stirring for a suitable time to produce a clear solution; d) add the drug to the solution of step c) with stirring for a suitable time; e) stir the solution of step d) for a suitable time and adjust the pH using one or more suitable pH adjusting agents; f) make the volume up to final batch size using water for injection; g) filter the solution of step f) using suitable filters (such as 0.2-micron size); h) fill the solution of step g) in appropriate size vials; i) stopper the vials using rubber stoppers; j) cap the vials using suitable seals (such as aluminum flip-off seals).

What is claimed:

1. A method for reducing or the non-surgical removal of body fat in an individual, the method comprising administering to the individual an injection pharmaceutical composition comprising:
a) a compound of the formula: 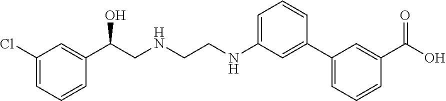 [SMILES: O=C(O)c1cccc(-c2cccc(NCCNC[C@H](O)c3cccc(Cl)c3)c2)c1]
or its pharmaceutically acceptable salts, esters, solvates, polymorphs, enantiomers, or mixtures thereof; and
b) at least one or more pharmaceutically acceptable excipients.

2. The method of claim 1, wherein the pharmaceutically acceptable salt is the hydrochloride salt.

3. The method of claim 1, wherein the composition comprises of from about 0.01% to about 40% of solabegron or its pharmaceutically acceptable salts, esters, solvates, polymorphs, enantiomers, or mixtures thereof.

4. The method of claim 1, wherein the composition is prepared by preparing a mixture comprising solabegron or its pharmaceutically acceptable salts, esters, solvates, polymorphs, enantiomers, or mixtures thereof, carrier and a pharmaceutically acceptable excipient.

5. The method according to claim 4, comprising the step of terminal sterilization or aseptic filtration.

6. The method according to claim 1, wherein the composition is suitable for administration via intramuscular or subcutaneous injection.

7. The method according to claim 1, wherein the composition is packed in a suitable container selected from a vial, ampoule, syringe, pen, auto-injector, cartridge.

8. The method according to claim 1, wherein the body fat is related to a condition selected from double chin disorder, benign symmetric lipomatosis, adiposis dolorosa, lipedema, and familial partial lipodystrophy.

9. The method of claim 1, wherein the therapeutically effective amount of solabegron or its pharmaceutically acceptable salts, esters, solvates, polymorphs, enantiomers, or mixtures thereof is administered within a plurality of treatment sessions.

10. The method of claim 1, wherein the therapeutically effective amount of solabegron or its pharmaceutically acceptable salts, esters, solvates, polymorphs, enantiomers, or mixtures thereof is administered within a plurality of treatment sessions with injection composition administered into adipose tissue of the said subject.

11. The method of claim 1, wherein said method is intended to reduce submental adipose tissue.

12. The method of claim 1, wherein the fat is reduced from a body part selected from the group consisting of the abdomen, chin, waist, arm, leg, knee, thigh, chest, breast, neck, face, buttock, lateral buttock, peri-orbital region, and intra-orbital region.

13. The method of claim 1, wherein the therapeutically effective amount of solabegron or its pharmaceutically acceptable salts, esters, solvates, polymorphs, enantiomers, or mixtures thereof is administered within a plurality of treatment sessions wherein each treatment session is spaced apart by at least 1 day.

14. The method of claim 1, wherein said plurality of treatment sessions with injection composition are spaced apart on the patient's body by about 0.3 cm.

15. A method for reducing or the non-surgical removal of body fat in an individual, the method comprising administering to the individual an injection pharmaceutical composition comprising:
a) solabegron or its pharmaceutically acceptable salts, esters, solvates, polymorphs, enantiomers, or mixtures thereof; and
b) at least one or more pharmaceutically acceptable excipients selected from the group comprising solvent, carrier, solubilizing agent, wetting agent, tonicity adjusting agent, pH adjusting agent, stabilizer, preservative, and antioxidant, and wherein the composition has a pH of about 3 to about 7.0.

16. The method of claim 15, wherein the solvents are selected from the group comprising aqueous and/or non-aqueous solvents.

17. The method of claim 15, wherein the pH adjusting agents are selected from the group comprising inorganic acids, organic acids, inorganic bases, and organic bases.

18. The method of claim 15, wherein the tonicity adjusting agents are selected from the group comprising sodium chloride, dextrose, mannitol, ascorbic acid, boric acid, citric acid, propylene glycol, sorbitol, and combinations thereof.

19. A kit comprising: a first container comprising a pharmaceutical composition according to claim 15, and optionally a second container comprising a suitable diluent or solvent.

20. A liquid pharmaceutical composition suitable for parenteral administration comprising:
a) about 0.001% to about 90% of solabegron,
b) about 0.01% to about 70% of one or more pH adjusting agents, and
c) one or more parenteral solvents.
